(19) United States
(12) Patent Application Publication
(10) Pub. No.: US 2007/0227538 A1
(43) Pub. Date: Oct. 4, 2007

(54) Method and device for controlling a ventilator

(76) Inventors: Bernd Scholler, Karlsruhe (DE); Matthias Schwaibold, Karlsruhe (DE)

Correspondence Address: Friedrich Kueffner, Suite 910, 317 Madison Avenue, New York, NY, 10017, US

(21) Appl. No.: 11/716,539

(22) Filed: Mar. 8, 2007

(30) Foreign Application Priority Data
Mar. 8, 2006 (DE) 10 2006 011 110.9

(51) Int. Cl. A61M 16/00; A62B 7/00

(52) U.S. Cl. 128/204.18; 128/204.21

(57) ABSTRACT

The device is used for ventilation, and the method serves the purpose of controlling a ventilator. Respiratory gas is supplied by a compressed gas source that can be connected to a patient interface. The compressed gas source is connected with a control system, and a measuring device is used to determine at least one parameter related to the flow of respiratory gas. The control system is provided with an analyzer for determining at least one event related to the flow of respiratory gas.

The invention concerns a method and a device for controlling a ventilator, in which at least two different pressure levels can be set for a respiratory gas supply system, and in which at least one ventilation parameter is measured and evaluated for controlling the ventilation pressure.

The invention also concerns a device for monitoring at least one ventilation parameter while respiratory gas is being supplied to a patient, which monitoring device has a sensor unit for detecting the behavior of the ventilation parameter as a function of time.

Large numbers of persons suffer from sleep disorders, which affect the well-being of these persons during the day and in some cases have an adverse effect on their quality of life. One of these sleep disorders is sleep apnea, which is treated primarily by CPAP therapy (CPAP=Continuous Positive Airway Pressure), in which a flow of respiratory gas is continuously supplied to the patient through a nasal mask. A hose connects the mask with a ventilator, which includes a blower that produces a gas flow with a positive pressure of 5 to 20 mbars.

The gas flow is supplied to the patient either at constant pressure or, to relieve the respiratory work of the patient, at a lower level during expiration. Although sleep apnea occurs for only short periods of time and constitutes only a small fraction of sleep, the blower runs for the entire period of sleep (night) in both methods, and this makes acceptance of sleep apnea treatment more difficult.

U.S. Pat. No. 5,245,995 A describes a CPAP ventilator that can be used for patients with sleep apnea. The respiratory gas is supplied to the patient by a breathing mask, and a compressed gas source is installed close to the ventilator. The compressed gas source can be controlled as a function of respiratory resistance.

EP 0 373 585 A describes a method for determining the respiratory resistance of a patient by means of ORM measurements. In this method, an oscillating volume flow with a low volume stroke is superimposed on the respiratory volume flow at a predetermined frequency. The periodic pressure variation that occurs at the same frequency can be used to obtain a measured value that is a function of the actual respiratory resistance.

U.S. Pat. No. 5,318,038 A describes a respiratory measurement to be carried out in the gas supply line with the use of a pneumotachygraph. EP 0 705 615 A describes a high-quality realization of a control system for a ventilator based on the performance of ORM measurements.

However, the prior-art methods and apparatus do not yet make it possible to carry out fast and simple apparatus-based support only when this is actually needed. This goal requires optimization of the automatic control of the ventilator and the use of suitable automatic control technology components. In particular, it is necessary to detect deviations from the patient's normal breathing activity at the earliest possible moment and to respond to these deviations by suitable automatic control of the ventilator.

Therefore, the objective of the present invention is to improve a method of the aforementioned type in such a way that the operation of the apparatus can be adapted as quickly as possible to the given ventilation state.

In accordance with the invention, at least one ventilation parameter is detected, and the behavior of at least one ventilation parameter as a function of time is detected and analyzed with respect to typical events.

A further objective of the present invention is to design a device of the aforementioned type in such a way that, with a simple technical design of the apparatus, changes in a given respiratory situation are promptly recognized.

The compressed gas source delivers respiratory gas to the patient. The flow and/or pressure of the respiratory gas delivered to the patient is determined at least periodically by at least one sensor unit. The sensor unit communicates with an analyzer.

The ventilator has an automatically controlled compressed gas source that can be connected with a patient interface, for example, a mask, a control system for the compressed gas source, and a measuring device for detecting at least one respiratory parameter. For example, the flow and/or pressure of respiratory gas delivered to the patient is determined at least periodically. The control system has an analyzer for determining at least one event.

The analyzer is set up in such a way that it determines, at least periodically, the respiratory cycle and/or the respiratory phase of the patient from the data recorded by the measuring device. In addition, the analyzer is suitable for determining inspiratory phases and expiratory phases. For example, a flow contour of an inspiration is determined from the flow signal of the inspiration. The analyzer is also designed to determine flow limitations, for example, in the flow contour of the inspiration.

A flow limitation is a partial obstruction of the upper airways which at least partially limits the flow of respiratory gas into the lung.

In accordance with the invention, an evaluation is made for each inspiration as to whether the present flow contour corresponds to that of a flow limitation, i.e., it has a distinct notch in the middle of the inspiration.

If this is the case, this flow curve of the inspiration is marked with a flattening marker. The flattening marker is temporary and can be recognized and evaluated by external instruments or by software.

If at least three breaths with flattening accumulate, a flattening event is recognized, which is likewise recognizable for other events by marking. This is stored in the compliance memory of the device and later makes it possible to provide detailed information about the success of treatment. In addition, this makes it possible not only to optimize the automatic control of the CPAP pressure, but also temporarily to deactivate a possibly active expiratory pressure drop to facilitate expiration in order to provide reliable treatment of residual obstructions.

The recognition is made by evaluation of the volume reduction of the present inspiration in percent with respect to the same inspiration after replacement of the convex flow contour components by a constructed connecting line, e.g., a straight line (corrected flow contour). This is not a comparison with “normal respiration” or “ideal respiration”, since the corrected flow contour also generally does not correspond to a physiological normal contour.

The recognition of flattening is robust compared to artifact-produced distortions of the flow curve. Another advantage of the invention is that only a single flattening index (area difference instead of width and height of the notch) needs to be computed, which, e.g., can also be computed in the polysomnography software for diagnostic nights. The flattening index is a value that represents the number and/or the extent of the flow limitations that arise due to partial obstruction of the upper airways. The process sequence is explained in detail below:

1. During inspiration

storage of the flow values (resolution preferably 100 Hz)

search for peak flow and peak flow position

integration of the flow values to inspiratory volume

at the end of the inspiration: discard implausible inspirations (too long, too short, peak flow too small, bordering on flow phase no breath), otherwise evaluation of the inspiration.

Evaluation of the Inspiration in Several Retrieval Steps:

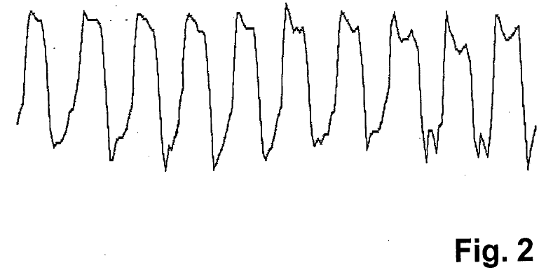

2. Creation of the corrected flow contour for the segment before the peak flow (see FIG. 2)

beginning with the first sampled value of the inspiration, end at the peak flow (“from front to back”); step width: preferably 5 values (20 Hz)

discard all sampled values up to the first value >⅓ □ peak flow

check for each value: if the present value is ABOVE the connecting line from the last value of the corrected flow contour to the peak flow (→concave): adopt the present value in the corrected flow contour; if the present value is BELOW the connecting line from the last value of the corrected flow contour to the peak flow (→convex): adopt the value of the line in the corrected flow contour.

3. Creation of the corrected flow contour for the segment after the peak flow

beginning with the last sampled value of the inspiration, end at the peak flow (“from back to front”); step width: 5 values (20 Hz)

check for each value: if the present value is ABOVE the connecting line from the last value of the corrected flow contour to the peak flow (→concave): adopt the present value in the corrected flow contour; if the present value is BELOW the connecting line from the last value of the corrected flow contour to the peak flow (→convex): adopt the value of the line in the corrected flow contour.

4. Form the flattening index

compute the percent reduction of the inspiratory volume of the breath with respect to the corrected flow contour as 100×(corrected flow contour−actual flow contour)/corrected flow contour

compute the flattening index as 3×the percent reduction. If the percent reduction >33, then the flattening index is always 100.

In the case of breaths with increased volume (hyperventilation, detection in the flow module), the flattening index is always 0.

5. Flattening detection (decision):

If the flattening index is above a certain threshold (e.g., 25), then the present breath is flow-limited. The temporary flattening marker is set.

If the temporary flattening marker is set, the flattening counter is increased by 1 as long as it is still <3.

If the temporary flattening marker is not set, the flattening counter is decreased by 1 as long as it is still >0.

A flattening event begins when the flattening counter=3 and end when it is <2.

Alternatively, it is possible to compute an inspiratory flow contour which gives the behavior of the respiratory flow for the given breath and the given patient if absolutely no obstruction of the upper airways were present.

This flow contour is preferably determined by deriving a typical or average flow curve from preceding, obviously unobstructed inspirations of the patient and calibrating it in amplitude and duration on the basis of a signal pattern that is related to the flow pattern at the beginning and the end of the current respiratory flow. This can be done, for example, by interpolation of the signal patterns.

Alternatively and likewise preferably, the obstruction-free flow contour is determined by recording a physical model for the behavior of the upper airways in the device, such that the parameters of the model are estimated from previous, obviously unobstructed inspirations of the patient. The model preferably contains differential equations on the behavior of collapsible tubes. On the basis of the model and the signal pattern of the present breath as input or output signal of the model, parameters are determined which are related to the pattern of respiratory effort and the variation of the cross section of the upper airways of the patient. The obstruction-free flow contour can be determined from these parameters.

The respiratory effort of the present breath is determined from the amplitude of the obstruction-free flow contour, either absolutely or relative to the preceding breaths. The severity of the flow limitation or a value related to the resistance of the upper airways is computed from the difference of the obstruction-free flow contour and the flow contour actually measured. When there is a change in the respiratory volume, this allows exact determination of the relative extent to which this change was caused by a change in respiratory effort or a change in airway resistance. Accordingly, the device can then optimally counteract the change in the respiratory volume by adjusting an average ventilation pressure when there is a change in resistance and a pressure difference between inspiration and expiration when there is a change in respiratory effort.

Furthermore, in accordance with the invention, a value related to the respiratory work can be determined from the parameters of respiratory effort and airway resistance.

The goal is to obtain quantitative information about the severity of the flow limitation in order to be able, for each respiratory event, to specify the exact percentage that was caused by obstruction and/or central factors.

The drawings show specific embodiments of the invention.

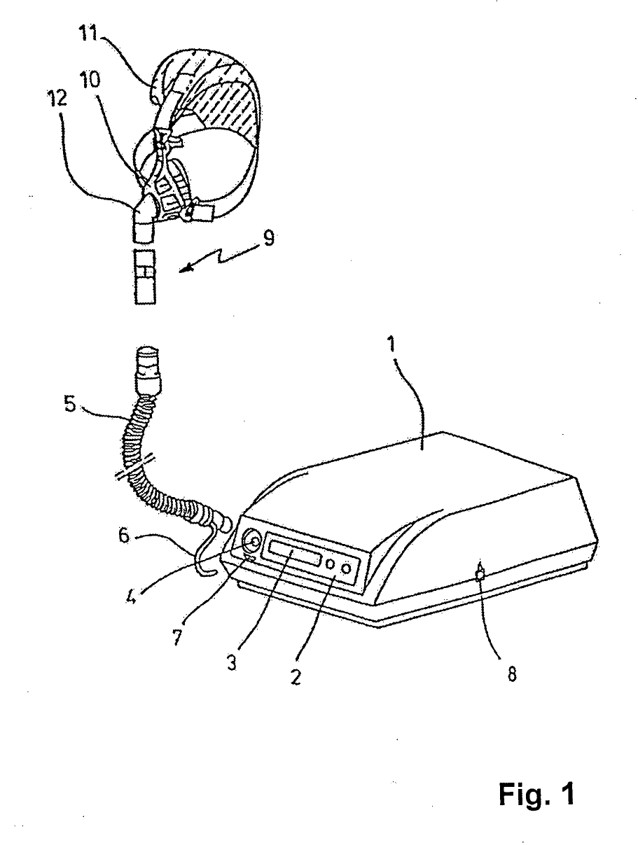

FIG. 1 is a schematic representation of a ventilator with a ventilation mask.

FIG. 2 shows a respiratory flow curve.

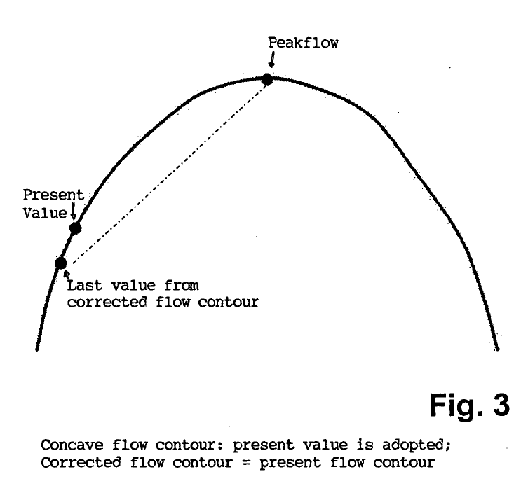

FIG. 3 shows the peak of a respiratory flow curve.

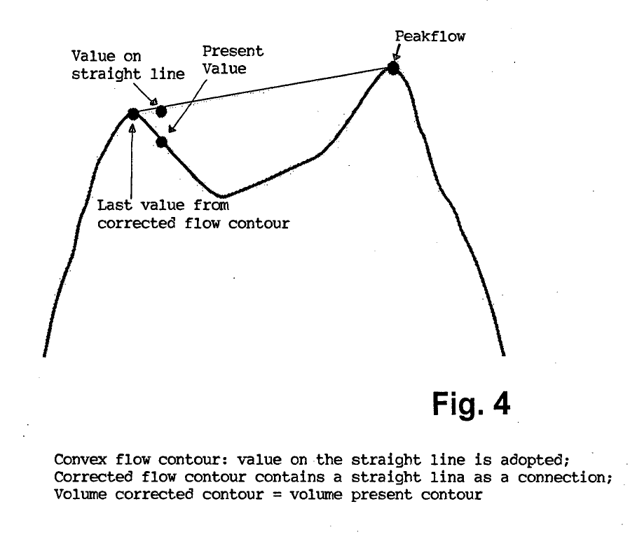

FIG. 4 shows a corrected respiratory flow curve.

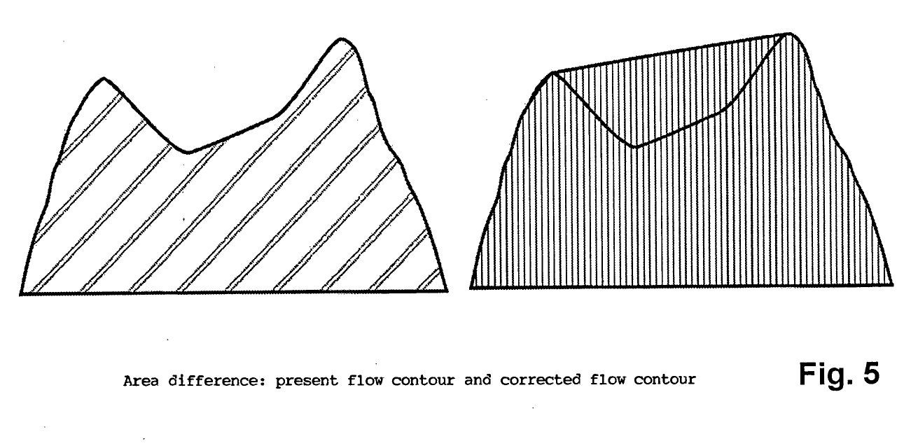

FIG. 5 shows the area difference between the determined and corrected respiratory flow curve.

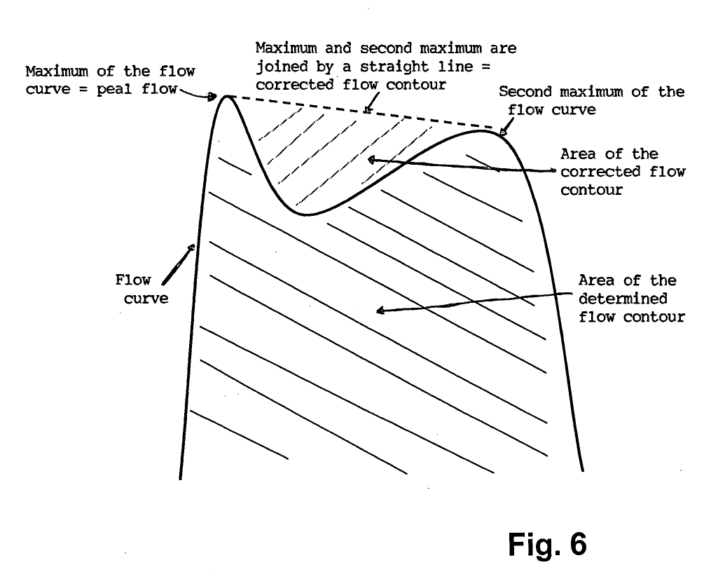

FIG. 6 shows a respiratory flow curve.

FIG. 1 shows the basic design of a ventilator. A respiratory gas pump is installed inside an apparatus housing 1, which has an operating panel 2 and a display 3. A connecting hose 5 is attached by a coupling 4. An additional pressure-measuring hose 6, which can be connected with the ventilator housing 1 by a pressure input connection 7, can run along the connecting hose 5. To allow data transmission, the ventilator housing 1 has an interface 8. An expiratory element 9 is installed in an expanded area of the connecting hose 5 that faces away from the apparatus housing 1.

FIG. 1 also shows a ventilation mask 10, which is designed as a nasal mask. The mask can be fastened on the patient's head by a head fastening device 11. A coupling device 12 is provided in the expanded region of the ventilator mask 10 that faces the connecting hose 5.

The fact that events can be identified by means of measured respiratory parameters is exploited in the device and the method of the invention.

### EXAMPLES OF SUCH EVENTS ARE

Mouth expiration, mouth breathing, leakage, swallowing, speaking, sneezing, coughing, increase in respiratory flow, decrease in respiratory flow, flattening of the respiratory flow, cessation of respiratory flow, increase in resistance, leakage, apnea, hypopnea, snoring, inspiration, expiration, interruption of breathing, increase in respiratory volume, decrease in respiratory volume, inspiratory “indentation” of the respiratory flow, inspiratory peak flow, decrease in the inspiratory flow after peak flow, second maximum of the inspiratory peak flow, increase in the pressure of the respiratory gas, decrease in the pressure of the respiratory gas, increase in the flow of the respiratory gas, decrease in the flow of the respiratory gas, increase in the volume of respiratory gas delivered, decrease in the volume of respiratory gas delivered.

A typical process sequence is carried out by designing the control system to perform CPAP, APAP, or bilevel ventilation, home ventilation, hospital ventilation, intensive ventilation, or emergency ventilation.

In one embodiment of the invention, the analyzer is designed to evaluate a flow pattern.

In another embodiment, the analyzer is designed to evaluate a flow contour. In addition, it is proposed that the analyzer be designed to evaluate pressure variation. In one variant of the method, the analyzer is designed to evaluate inspiration phases.

Furthermore, it is possible to design the analyzer to evaluate expiration phases. A simple evaluation principle consists in designing the analyzer to evaluate amplitude values. In addition, it is also possible to design the analyzer to evaluate output values.

When events are stored and evaluated, it is possible to refine the quality of the response of the device by a self-learning system.

The ventilator has a compressed gas source that can be connected to a user interface, a control system for the compressed gas source, and a measuring device for determining at least one respiratory parameter. The control system is provided with an adaptation device for varying the pressure made available by the compressed gas source as a function of an analysis of the measured respiratory parameter. The control system has an analyzer for detecting at least one event.

The ventilator delivers an essentially positive respiratory gas pressure, which can be in the range of 0 to 80 mbars, with an electrically controlled respiratory gas source.

The ventilator preferably has an automatic controller for controlling the respiratory gas supply according to the events detected by the measuring device for the purpose of setting a suitable pressure level, which can be in the range of 0 hPa to 80 hPa.

In one embodiment of the invention, the automatic controller increases the pressure level in at least one operating mode and reduces the pressure level in at least one other operating mode, such that the automatic controller considers at least one event, and the reduction of the pressure level occurs essentially in unison with the expiratory phase of a user. In this connection, the automatic controller usually does not allow the pressure to fall below 2 mbars. In at least one other operating mode, the pressure level is reduced in unison with the expiratory phase of a user.

FIG. 2 shows a typical respiratory flow curve. FIG. 3 shows a greatly magnified peak of the respiratory flow curve according to FIG. 2. According to step No. 1 of the process sequence explained above, the present flow values are stored, and a search is made for the peak flow value on the basis of the stored and analyzed values. A current value of the flow is adopted as the maximum value in the evaluation as long as an increase is detected relative to the last value. After the maximum flow value has been determined in this way, step No. 2 of the process sequence is used to determine the corrected flow contour. The connecting line explained in connection with step No. 2 is drawn as a broken line in FIG. 3.

FIG. 4 shows a flow contour pattern with two successive peak values, such that the second peak value constitutes the actual maximum value. In regard to the determination of the corrected flow contour, for the region between the first and second peak value, the notch in the signal pattern is replaced by a straight line between the two peak values.

For a flow contour pattern of the type shown in FIG. 4, FIG. 5 compares the areas of the actual pattern of the flow contour and of the pattern for the corrected flow contour.

FIG. 6 illustrates a signal pattern, according to which, in a departure from the representation in FIG. 4, the first determined maximum value constitutes the actual maximum value, but after this maximum value, a steady decrease of the signal does not occur, but rather the signal passes through an intermediate minimum. According to step No. 3 of the process sequence, a correction is also made here by replacing the region of the signal notch by a connecting line between the first and second maximum values.

For the patterns according to FIGS. 4 and 6, it is basically the case that, even when additional intermediate maxima occur, a straight line is formed between the actual signal maximum and the second largest maximum value.

For patterns that differ from those shown in FIGS. 4 and 6, it is also basically the case that possible intermediate minima are generally replaced by straight lines between adjacent maxima. If necessary, a curve that has already been corrected is corrected again if a maximum farther to the right in the plane of the drawing is greater than a maximum that has already been considered. Basically, signal patterns that are convex in a direction of view from the lower axis in the plane of the drawing towards the signal curve are replaced by straight lines.

1. A ventilator, which has a compressed gas source that can be connected to a patient interface to deliver respiratory gas, a control system for the compressed gas source, and a measuring device for determining at least one parameter related to the flow of respiratory gas, wherein the control system has an analyzer for determining at least one event that is related to the flow of respiratory gas.

2. A ventilator in accordance with claim 1, wherein the analyzer is capable of identifying an inspiration and/or an expiration.

3. A ventilator in accordance with claim 1, wherein the analyzer is capable of identifying a flow contour of the inspiration.

4. A ventilator in accordance with claim 1, wherein the analyzer is capable of identifying an inspiratory peak flow.

5. A ventilator in accordance with claim 1, wherein the analyzer is capable of removing convex components of the inspiratory flow contour and replacing them with corrected flow contour segments.

6. A ventilator in accordance with claim 1, wherein the analyzer is capable of removing concave components of the inspiratory flow contour and replacing them with corrected flow contour segments.

7. A ventilator in accordance with claim 1, wherein the corrected flow contour segments consist of straight line segments.

8. A ventilator in accordance with claim 1, wherein the analyzer is capable of identifying a flow limitation.

9. A ventilator in accordance with claim 1, wherein the analyzer is capable of identifying the flow limitation on the basis of area or volume ratios.

10. A method for controlling a ventilator, in which a compressed gas source that supplies respiratory gas is connected with a patient interface, at least one parameter that is related to the flow of respiratory gas is determined by a measuring device, and corresponding measured values are transmitted to the control system for the compressed gas source, wherein an analyzer that interacts with the control system determines at least one event that is related to the flow of respiratory gas.

11. A method in accordance with claim 10, wherein the analyzer identifies an inspiration and/or an expiration.

12. A method in accordance with claim 10, wherein the analyzer identifies a flow contour of the inspiration.

13. A method in accordance with claim 10, wherein the analyzer identifies an inspiratory peak flow.

14. A method in accordance with claim 10, wherein the analyzer removes convex components of the inspiratory flow contour and replaces them with corrected flow contour segments.

15. A method in accordance with claim 10, wherein the analyzer removes concave components of the inspiratory flow contour and replaces them with corrected flow contour segments.

16. A method in accordance with claim 10, wherein the flow contour segments to be corrected are replaced by straight line segments.

17. A method in accordance with wherein the analyzer identifies a flow limitation.

18. A method in accordance with claim 10, wherein the analyzer identifies the flow limitation on the basis of area or volume ratios.

19. A method in accordance with claim 10, wherein an inspiratory flow contour is computed which gives the behavior of the respiratory flow for the given breath and the given patient if absolutely no obstruction of the upper airways were present.

20. A method in accordance with claim 10, wherein an inspiratory flow contour is determined by deriving a typical or average flow curve from preceding, obviously unobstructed inspirations of the patient and calibrating it in amplitude and duration on the basis of a signal pattern that is related to the flow pattern at the beginning and the end of the current respiratory flow.

21. A method in accordance with claim 20, wherein an interpolation of the signal patterns is carried out.

22. A method in accordance with claim 10, wherein an obstruction-free flow contour is determined by recording a physical model for the behavior of the upper airways in the device, such that the parameters of the model are estimated from previous, obviously unobstructed inspirations of the patient.

23. A method in accordance with claim 10, wherein the respiratory effort of the present breath is determined from the amplitude of the obstruction-free flow contour, either absolutely or relative to the preceding breaths.

24. A method in accordance with claim 10, wherein the severity of the flow limitation or a value related to the resistance of the upper airways is computed from the difference of the obstruction-free flow contour and the flow contour actually measured.

25. A method in accordance with claim 10, wherein a value related to the respiratory work is determined from the parameters of respiratory effort and airway resistance.
